(19) United States
(12) Patent Application Publication
(10) Pub. No.: US 2020/0030489 A1
(43) Pub. Date: Jan. 30, 2020

(54) ONE-STEP FORMED GEL FIBER COMPOSITE SCAFFOLD MATERIAL AND PREPARATION METHOD AND USE THEREOF

(71) Applicant: NATIONAL CENTER FOR NANOSCIENCE AND TECHNOLOGY, Beijing (CN)

(72) Inventors: Xinxiao HAN, Beijing (CN); Dong HAN, Beijing (CN)

(21) Appl. No.: 16/338,094

(22) PCT Filed: Jan. 25, 2017
(86) PCT No.: PCT/CN2017/072608

§ 371 Date: Mar. 29, 2019

(30) Foreign Application Priority Data
Sep. 29, 2016 (CN) 201610866896.0

(51) Int. Cl. A61L 27/18; A61L 27/20

(52) U.S. Cl. CPC A61L 27/18; A61L 27/24; A61L 27/20

(57) ABSTRACT

A one-step formed gel fiber composite scaffold material can be prepared by preparing an electrospun film through a high-voltage electrostatic method and soaking the electrospun film into a phase separation solution to obtain the gel fiber composite scaffold material. The obtained composite scaffold material is an extracellular matrix simulated composite material and can be modified by adding multiple natural biomaterials, so that the composite material is closer to an extracellular matrix in terms of components

### CROSS-REFERENCE TO RELATED APPLICATION

This application claims priority to Chinese Patent Application No. 201610866896.0, filed on Sep. 29, 2016, which is incorporated herein by reference in its entirety.

### TECHNICAL FIELD

The present invention relates to a one-step formed gel fiber composite scaffold material and preparation method and use thereof.

BACKGROUND ART

The extracellular matrix is a highly hydrated fibrous network gel structure composed of proteins and polysaccharides, which is distributed in the extracellular space and secreted by cells. The structure and component proportions of the extracellular matrix are slightly different in different tissues, but the basic composition thereof is similar. The extracellular matrix has a fiber network consisted essentially of collagen/elastin, and a gel-like matrix formed by non-collagen glycoproteins, amino glycans, and proteoglycans, and provides attachment sites for surrounding cells. The extracellular matrix not only participates in the maintenance of tissue structures, but also affects the basic life activities of the cells, such as survival form, function, metabolism, proliferation, differentiation, migration and the like. The important influence of extracellular matrix on various pathophysiological activities of cells has attracted the attention of the biological field for a long time, and many extracellular matrix material products have also been produced. However, the preparation process of most products is cumbersome, the product is of high price, and it is impossible to carry out extensive fundamental research and applications.

### SUMMARY OF THE INVENTION

The object of the present invention is to overcome the shortcomings of the prior art, such as the cumbersome preparation processes, high price and low yield of the products, and to provide a one-step formed gel fiber composite scaffold material and preparation method and use thereof, in order to achieve its application in different biological fundamental research.

By the research of the invention, it is found that the preparation processes for the extracellular matrix-imitating materials according to the prior art are complicated, the prepared components are relatively simple, and the gel fiber composite material cannot be formed at one step. Generally, only the fiber structure or a single component is taken into consideration in common materials, while natural extracellular matrix products have high cost, low yield, and less basic application. High-voltage electrospinning technology is a common method for producing micro- or nano-scaled fiber materials. Type I collagen and gelatin are commonly used materials for the pretreatment of the substrate for cell culture, and both have the function of promoting cell adhesion and spreading.

Based on the above findings, the present invention provides a one-step formed gel fiber composite scaffold material prepared based on high-voltage electrospinning technique and phase separation technique with low cost.

The technical solutions of the present invention are as follows:

The one-step formed gel fiber composite scaffold material is prepared by the following steps: preparing an electrospun film through a high-voltage electrostatic method; and then soaking the electrospun film into a phase separation solution to obtain the one-step formed gel fiber composite scaffold material.

The electrospun film undergoes phase separation under the action of the phase separation solution to form a fibrous gel network structure.

The raw materials for preparing the electrospun film comprise polycaprolactone, gelatin, and type I collagen; wherein preferably, the mass ratio of polycaprolactone, gelatin, and type I collagen is 10-20:10-20:5-10, more preferably 10:10:5.

The solvent for preparing the electrospinning solution is preferably trifluoroethanol.

The conditions for preparing the electrospun film are as follows: voltage of 15 to 20 kV, electrospinning distance of 10 to 20 cm, and electrospinning time of 10 to 60 min.

The electrospinning needle can be a 12G type electrospinning needle.

The phase separation solution is a mixed solution of water-soluble carbodiimide and N-hydroxysuccinimide, wherein the molar concentrations of the water-soluble carbodiimide and N-hydroxysuccinimide are preferably 0.05 to 0.1 M, respectively, and more preferably both are 0.05 M.

Alternatively, the phase separation solution is a mixed solution of the above mixed solution of water-soluble carbodiimide and N-hydroxysuccinimide with an aqueous solution of sodium hyaluronate.

Specifically, the preparation method of the above-mentioned one-step formed gel fiber composite scaffold material comprises:

1) preparation of an electrospinning solution: dissolving polycaprolactone, gelatin and type I collagen in trifluoroethanol, respectively, and magnetically stirring for 3 to 6 hours to give a polycaprolactone solution with a mass fraction of 10 to 20%, a gelatin solution with a mass fraction of 10 to 15%, and a type I collagen solution with a mass fraction of 5 to 10%;

2) high-voltage electrospinning: using the polycaprolactone solution, gelatin solution and type I collagen solution prepared in the step 1) as raw materials to perform high-voltage electrospinning to obtain an electrospun film;

wherein the conditions for electrospinning are as follows: the voltage is 15 to 20 kV, the electrospinning distance is 10 to 20 cm, the electrospinning needle is a 12G type electrospinning needle, and the electrospinning time is 10 to 60min;

3) preparation of a phase separation solution: separately preparing 1M water-soluble carbodiimide solution and 1M N-hydroxysuccinimide solution, completely mixing the two solutions in equal volume, and then diluting 10 times to give the phase separation solution;

Alternatively, further mixing the prepared phase separation solution with a sodium hyaluronate aqueous solution having a mass fraction of 1% in a volume of 1:1; and

4) soaking the electrospun film obtained in the step 2) in the phase separation solution prepared in the step 3) for at least 20 minutes, and washing with pure water to obtain the one-step formed gel fiber composite scaffold material.

The present invention also includes a one-step formed gel fiber composite scaffold material prepared by the method.

The present invention also includes the use of the one-step formed gel fiber composite scaffold material described above in a cell culture substrate, a transplant material and the like.

The composite scaffold material of the present invention is an extracellular matrix-imitating composite material, and can also be modified by adding multiple natural biomaterials, so that the composite material is more similar to an extracellular matrix in terms of components.

The advantages or beneficial effects of the present invention lie in that, compared with other existing extracellular matrix materials, the material of the present invention is simpler in preparation process, easier to store and transport, and lower in cost, which is beneficial to wide use in fundamental research.

The high-voltage electrospun film prepared by the present invention has high biocompatibility and high elasticity modulus, fits with the surface of skin and organs well, and is more suitable for use in cell culture substrate and transplant materials.

### BRIEF DESCRIPTION OF THE DRAWINGS

FIG. 1 shows an electronograph characterizing the surface morphology of the experimental sample of Experimental Example 1.

FIG. 2 shows a stress-strain curve of the experimental sample of Experimental Example 1.

### SPECIFIC MODES FOR CARRYING OUT THE EMBODIMENTS

The following examples are intended to illustrate the invention but are not intended to limit the scope of the invention. The specific techniques or conditions being not indicated in the examples are in accordance with the techniques or conditions described in the literatures in the art, or in accordance with the product specifications. The reagents or instruments used without indicating the manufacturer are all conventional products that are commercially available.

Polycaprolactone, having a molecular weight of 80,000, was purchased from sigma/vetec;

Gelatin was purchased from sigma/vetec;

Type I collagen was purchased from Sichuan Mingrang Biotechnology Co., Ltd.;

Trifluoroethanol was purchased from Aladdin (Shanghai Jingchun Biochemical Technology Co., Ltd.);

High-voltage power supply was purchased from Dongwen High-Voltage Power Co., Ltd.;

Water-soluble carbodiimide was purchased from Aladdin (Shanghai Jingchun Biochemical Technology Co., Ltd.);

N-hydroxysuccinimide was purchased from Aladdin (Shanghai Jingchun Biochemical Technology Co., Ltd.).

Sodium hyaluronate was purchased from Aladdin (Shanghai Jingchun Biochemical Technology Co., Ltd.), and prepared by microbial fermentation.

### EXAMPLE 1

The preparation method of one-step formed gel fiber composite scaffold material comprises:

1) preparation of electrospinning solution: polycaprolactone, gelatin and type I collagen were dissolved in trifluoroethanol, respectively, and magnetically stirred for 3 to 6 hours to give a polycaprolactone solution with a mass fraction of 10%, a gelatin solution with a mass fraction of 10%, and a type I collagen solution with a mass fraction of 5%;

2) high-voltage electrospinning: the 10% polycaprolactone solution, 10% gelatin solution, and 5% type I collagen solution prepared in the step 1) were used as raw materials to perform high-voltage electrospinning to obtain an electrospun film;

wherein the conditions for electrospinning were as follows: voltage of 15 to 20 kV, electrospinning distance of 10 to 20 cm, 12G-type electrospinning needle, and electrospinning time of 10 min, 30 min and 60 min, respectively;

3) preparation of phase separation solutions: 1 M water-soluble carbodiimide solution and 1 M N-hydroxysuccinimide solution were prepared, respectively; the two solutions were completely mixed, and then diluted 10 times to give a phase separation solution; and

4) the electrospun film obtained in the step 2) was soaked in the phase separation solution prepared in the step 3) for at least 20 minutes, and washed with pure water to obtain the one-step formed gel fiber composite scaffold material.

### EXAMPLE 2

The preparation method of one-step formed gel fiber composite scaffold material was the same as that of Example 1 except that the phase separation solution used in the step 3) was different.

The phase separation solution of this Example was as follows: 1M water-soluble carbodiimide solution and 1 M N-hydroxysuccinimide solution were prepared, respectively; the two solutions were completely mixed in equal volume, and diluted 10 times to obtain a liquid A.

A sodium hyaluronate aqueous solution having a mass fraction of 1% was prepared as a liquid B. The liquid A and the liquid B were mixed in a volume of 1:1 to obtain a phase separation solution, and then phase separation was carried out on the film.

### EXAMPLE 3

The preparation method of one-step formed gel fiber composite scaffold material was the same as that of Example 1 except that a polycaprolactone solution with a mass fraction of 20%, a gelatin solution with a mass fraction of 20%, and a type I collagen solution with a mass fraction of 10% were separately prepared as the electrospinning solutions in the step 1).

### EXAMPLE 4

The preparation method of one-step formed gel fiber composite scaffold material was the same as that of Example 1 except that the preparation of phase separation solution in the step 3) included: preparing 1 M water-soluble carbodiimide solution and 1 M N-hydroxysuccinimide solution, respectively, completely mixing the two solutions in equal volume, and diluting 5 times to give a phase separation solution.

### EXPERIMENTAL EXAMPLE

### Characterization of Surface Morphology of the Materials

Experimental samples: the electrospun film obtained in the step 2) of Example 1 (before phase separation) and the one-step formed gel fiber composite scaffold material obtained in the step 4) (after phase separation).

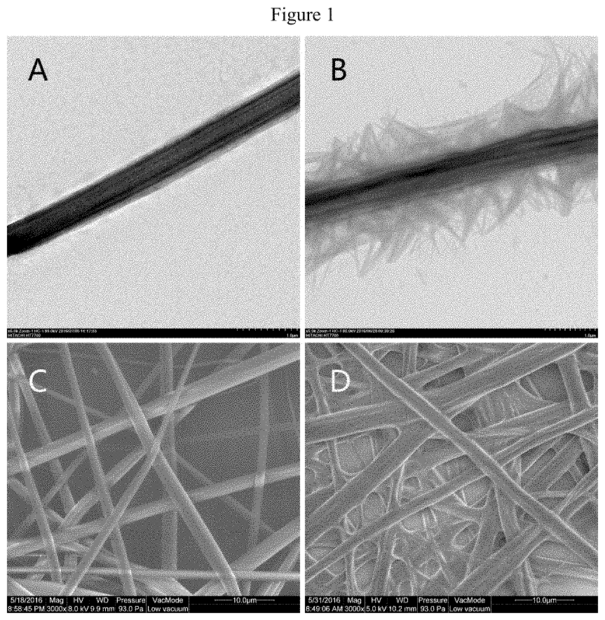

The surface morphology of the experimental samples was characterized and measured by electron microscopy: the instrument was a field emission environment scanning electron microscope (FEI, Quanta 200 FEG); 120KV transmission electron microscope (Hitachi, HT7700), the results were shown in FIG. 1, wherein:

(A) was a transmission electron microscope image of the electrospun film (before phase separation);

(B) was a transmission electron microscope image of the one-step formed gel fiber composite scaffold material (after phase separation); it can be seen that a gel-like polymer network was formed on the surface after phase separation of the high-voltage electrospun film;

(C) was an environmental scanning electron microscope image of the electrospun film (before phase separation); and

(D) was an environmental scanning electron microscope image of the one-step formed gel fiber composite scaffold material (after phase separation); the gel-like substance whose surface became unsmooth was observed after the high-voltage electrospinning and phase separation.

The film thickness of the experimental sample was measured by a step profiler which was a contact-type surface morphology measuring instrument (BRUKER, Dektak-XT), and the results were shown in Table 1. It can be seen that the thickness of the same fiber film was greatly reduced after phase separation.

TABLE 1

| | | one-step formed gel fiber |
|---|---|---|
| | electrospun film | composite scaffold material |
| Time | (before phase separation) | (after phase separation) |
| (minutes) | thickness (micron) | thickness (micron) |
| 10 | 8 | 3 |
| 30 | 36 | 12 |
| 60 | 50 | 16 |

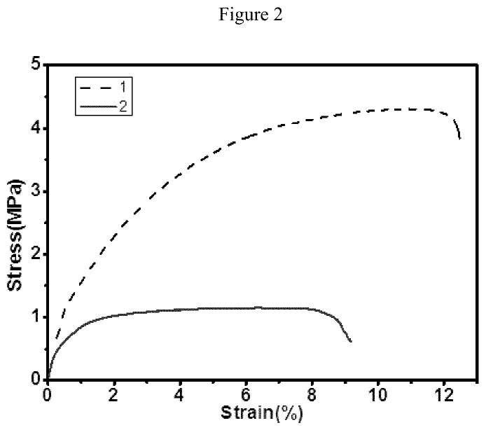

The elasticity modulus of the experimental sample was measured by a dynamic mechanical property analyzer: the instrument was a dynamic mechanical property analyzer (TA, DMA-Q800), quasi-static tensile test, and the results were shown in FIG. 2.

The abscissa Strain in FIG. 2 represents the percentage change of stress, the ordinate Stress represents the stress value, the solid line “-2” represents the stress-strain curve of the electrospun film (before phase separation); and the dotted line “--1” represents the stress-strain curve of the one-step formed gel fiber composite scaffold material (after phase separation). After calculation, the elasticity modulus of the fiber film before phase separation was 148.23±21.68 MP; and the elasticity modulus of the fiber film after phase separation was 180.55±60.46 MPa. It can be seen that the elasticity modulus of the same fiber film increases remarkably after phase separation.

Although the present invention has been described in detail with reference to the preferred embodiments of the present invention, it will be apparent to a person skilled in the art that modifications or improvements can be made thereto based on the present invention. Therefore, such modifications or improvements made without departing from the spirit of the invention are intended to be within the scope of the present invention.

### INDUSTRIAL APPLICABILITY

The invention provides a one-step formed gel fiber composite scaffold material and preparation method and use thereof. The gel fiber composite material can be rapidly prepared by the method of the present invention, and as compared with other existing extracellular matrix materials, the material of the present invention is simpler in preparation process, easier to store and transport, and lower in cost, which is beneficial to wide use in fundamental research. The gel fiber composite scaffold material of the present invention has high biocompatibility and high elasticity modulus, fits with the surface of skin and organs well, and is more suitable for use in cell culture substrate and transplant materials. The present invention has broad application prospects and good industrial applicability in the fields of biotechnology and the like.

1. A preparation method of a one-step formed gel fiber composite scaffold material, comprising: preparing an electrospun film through a high-voltage electrostatic method; and then soaking the electrospun film into a phase separation solution to obtain the gel fiber composite scaffold material; wherein the raw materials for preparing the electrospun film include polycaprolactone, gelatin, and type I collagen.

2. The preparation method according to claim 1, wherein the mass ratio of polycaprolactone, gelatin and type I collagen as the raw materials for preparing the electrospun film is 10-20:10-20:5-10; preferably 10:10:5.

3. The preparation method according to claim 1, wherein the solvent for preparing the electrospinning solution is trifluoroethanol.

4. The preparation method according to claim 1, wherein the conditions for preparing the electrospun film are as follows: voltage of 15 to 20 kV, electrospinning distance of 10 to 20 cm, and electrospinning time of 10 to 60 min.

5. The preparation method according to claim 1, wherein the phase separation solution is a mixed solution of water-soluble carbodiimide and N-hydroxysuccinimide; wherein the molar concentrations of the water-soluble carbodiimide and N-hydroxysuccinimide are preferably 0.05 to 0.1 M, respectively, and more preferably both are 0.05 M.

6. The preparation method according to claim 5, wherein the phase separation solution further contains sodium hyaluronate.

7. The preparation method according to claim 1, comprising:
1) preparation of an electrospinning solution: dissolving polycaprolactone, gelatin and type I collagen in trifluoroethanol, respectively, and magnetically stirring for 3 to 6 hours to prepare a polycaprolactone solution with a mass fraction of 10 to 20%, a gelatin solution with a mass fraction of 10 to 15%, and a type I collagen solution with a mass fraction of 5 to 10%;
2) high-voltage electrospinning: using the polycaprolactone solution, gelatin solution and type I collagen solution prepared in the step 1) as raw materials to perform high-voltage electrospinning to obtain an electrospun film;
wherein the conditions for electrospinning are as follows: voltage of 15 to 20 kV, electrospinning distance of 10 to 20 cm, 12G-type electrospinning needle, and electrospinning time of 10 to 60min;
3) preparation of a phase separation solution: separately preparing 1M water-soluble carbodiimide solution and 1M N-hydroxysuccinimide solution, completely mixing the two solutions in equal volume, diluting 10 times to give the phase separation solution;
alternatively, further mixing the prepared phase separation solution with a sodium hyaluronate aqueous solution having a mass fraction of 1% in a volume of 1:1; and
4) soaking the electrospun film obtained in the step 2) in the phase separation solution prepared in the step 3) for at least 20 minutes, and washing with pure water to obtain the gel fiber composite scaffold material.

8. A material comprising a one-step formed gel fiber composite scaffold material prepared by the method of claim 1.

9. A cell culture substrate comprising the material according to claim 8.

10. A transplant material comprising the material according to claim 8.
